(19) United States
(12) Patent Application Publication
(10) Pub. No.: US 2025/0006334 A1
(43) Pub. Date: Jan. 2, 2025

(54) SIROLIMUS PHARMACOKINETICS GUIDED AND MODEL INFORMED PRECISION DOSING

(71) Applicant: Children's Hospital Medical Center, Cincinnati, OH (US)

(72) Inventors: Tomoyuki Mizuno, Mason, OH (US); Denise Adams, Terrace Park, OH (US); Alexander Vinks, Cincinnati, OH (US); Adrienne Hammill, Cincinnati, OH (US)

(21) Appl. No.: 18/746,610

(22) Filed: Jun. 18, 2024

Related U.S. Application Data
(60) Provisional application No. 63/523,776, filed on Jun. 28, 2023.

(51) Int. Cl. G16H 20/10; G16H 10/40

(52) U.S. Cl. CPC G16H 20/10; G16H 10/40

(57) ABSTRACT

A personalized sirolimus dosing regimen may be determined and implemented based on individual-specific data for the individual who is being treated. This data may include hematocrit, co-medications, fever, growth, maturation, and the individual's sirolimus trough concentration. This data may be obtained as part of a method which includes using the individual-specific data to refine a sirolimus clearance model for the individual, and generating a personalized sirolimus dose for the individual using the refined sirolimus clearance model. Once has been generated, the personalized sirolimus dose may be administered to the individual.

### CROSS-REFERENCE TO RELATED APPLICATIONS

This application is a non-provisional U.S. application which claims priority to U.S. Provisional Application Ser. No. 63/523,776 filed Jun. 28, 2023, the contents of which is incorporated by reference in its entirety, for all purposes.

### STATEMENT REGARDING FEDERALLY SPONSORED RESEARCH

This invention was made with government support under FD003712 awarded by the National Institutes of Health. The government has certain rights in the invention.

### BACKGROUND

A physician's decision to start a patient on a medication-based treatment regimen involves development of a dosing regimen for the medication to be prescribed. Different dosing regimens will be appropriate for different patients having differing patient factors. By way of example, dosing quantities, dosing intervals, treatment duration and other variables may be varied. Although a proper dosing regimen may be highly beneficial and therapeutic, an improper dosing regimen may be ineffective or deleterious to the patient's health. Further, both under-dosing and over-dosing generally results in a loss of time, money and/or other resources, and increases the risk of undesirable outcomes. These issues are particularly acute regarding sirolimus, which exhibits large inter- and intra-patient variability in its disposition, and for pediatric populations, for which dosing information is very limited. Accordingly, there is a need for technology which can allow clinicians to more effectively determine and apply the proper dosing in a medication based treatment regimen, especially in the case of treating a pediatric patient using a regimen that includes administration of sirolimus.

### BRIEF SUMMARY

The technology disclosed herein may be applied in the personalization of dosing during a treatment regimen which includes administration of sirolimus. For example, the disclosed technology may be used to implement a method of determining and implementing a personalized sirolimus dosing regimen for an individual in need thereof. Such a method may comprise providing an initial sirolimus dose to the individual, and obtaining individual-specific data. This individual-specific data may comprise hematocrit, co-medications, fever, growth, maturation and sirolimus trough concentration. In such a method, this individual-specific data may be used to refine a sirolimus clearance model for the individual. The refined sirolimus clearance model may, in turn, be used to generate a personalized sirolimus dose for the individual. The personalized sirolimus dose may then be administered to the individual.

Other applications of the disclosed technology, including in the form of systems or computer readable media corresponding to methods such as described above. Additional benefits and advantages of the disclosed technology will be also apparent in view of the following description and accompanying figures. Accordingly, the described method should be understood as being illustrative only and should not be treated as limiting.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 illustrates a precision dosing method which may be implemented based on this disclosure;

FIG. 2 illustrates an exemplary result of customizing sirolimus dosing based on age and maturation to achieve a target concentration; and

FIG. 3 illustrates an exemplary result of customizing sirolimus dosing based on age and maturation to achieve a target concentration.

### DETAILED DESCRIPTION

Disclosed herein are techniques which may be used for determining precision dosing for sirolimus, a compound which may be used as a targeted therapy in pediatric populations. In this disclosure, concrete examples of sirolimus precision dosing in patients suffering acute lymphoblastic leukemia (ALL) are provided as a means to illustrate how the disclosed techniques may be applied in practice. However, it should be understood that these examples are intended to be illustrative only, and that the disclosed techniques may be applied in other context as well, such as where sirolimus is used for purposes such as the treatment of other types of cancers, or for it other effects such as an immunosuppressant. Similarly, in some aspects, the disclosed techniques may also be used with compounds other than sirolimus, such as with everolimus, another drug which can be used in cancer treatment. Accordingly, the examples provided here should not be treated as implying limitations on the protection provided by this document, or by any other related document.

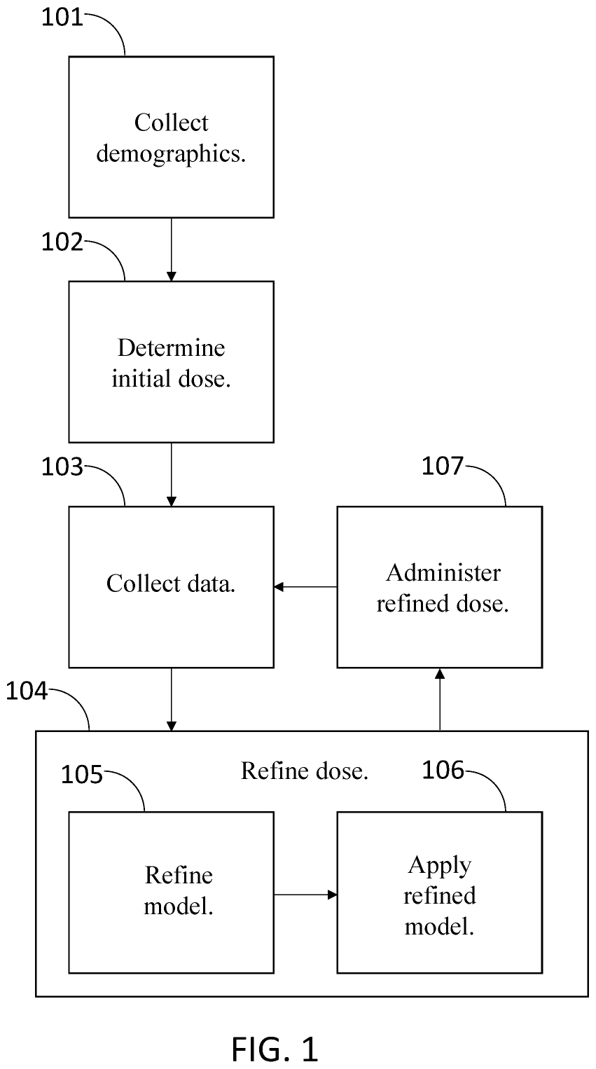

Turning now to the figures, FIG. 1 illustrates a precision dosing method which may be implemented based on this disclosure. Initially, in the method of FIG. 1, demographic information for a patient would be collected 101. This may be performed by, for example, automatically retrieving information such as the patient's weight, height, sex and age (including gestational age, if a course of treatment is to be started shortly after birth) from an electronic medical record corresponding to the patient. Alternatively, this information may be obtained through direct measurement (e.g., weighing the patient to determine his or her weight), through inquiries (e.g., asking the parent or guardian of a pediatric patient to provide his or her age), or through a combination of one or more of the foregoing methods (e.g., obtaining the patient's weight and height through direct measurement, while obtaining the patient's age through either an inquiry or through retrieving it from an electronic medical record system). Other approaches to obtaining demographic information will be immediately apparent to, and could be used to substitute for, or supplement, those described by one of ordinary skill in the art in light of this disclosure.

However it was collected 101, once the patient's demographic data was available, it may be used to determine 102 an initial dose for the compound to be administered, such as sirolimus. This may be done, for example, by using stored data which indicates a recommended dose for achieving a target concentration range based on the demographic data collected 101 for the patient. An example of this type of data is provided below in table 1, which illustrates doses (measured in mg/m², and administered twice daily) based on the body surface area and age of patients up to two years old for achieving concentrations of sirolimus.

TABLE 1
Exemplary age-based doses for sirolimus.

Target trough concentration range

| | 10-15 ng/ml | 5-10 ng/ml |
|---|---|---|
| Age group (months) | Dose | Dose |
| 0-1 | 0.4 | 0.25 |
| 1-2 | 0.5 | 0.30 |
| 2-3 | 0.6 | 0.37 |
| 3-4 | 0.7 | 0.45 |
| 4-6 | 0.9 | 0.55 |
| 6-9 | 1.1 | 0.70 |
| 9-12 | 1.3 | 0.85 |
| 12-24 | 1.6 | 1.0 |

After therapy has been initiated using the initial dose, data may be collected 103 on the impact of the therapy. This may be done, for example, by, 7-14 days after the start of therapy, taking a pre-dose trough blood sample which could then be sent to a lab to determine a sirolimus blood concentration. Alternatively, in some cases additional samples may be taken. For example, in a case where an abbreviated area under the curve PK consult is ordered, data collection 103 may include taking three whole blood samples: a first sample taken 15-30 minutes before a dose, a second sample taken 45-75 minutes after the dose, and a third sample taken 150-210 minutes after the dose. A laboratory could then process the sample(s), and the sirolimus concentration could be reported back. For example, in a case where an application for recommending individualized sirolimus doses was implemented based on this disclosure, the lab could report the results back through an API exposed by a web portal supported by the application, or could send the results back through an external communication channel (e.g., secure email) to the user of the application, and that user could then input the results to the application himself or herself.

Following data collection 103, in a method such as shown in FIG. 1, the collected data may be used to refine 104 the patient's dose. For the purpose of illustration, this dose refinement 104 is depicted in FIG. 1 as being split into two substeps, refining 105 a model and applying 106 the refined model. Turning to the first of those substeps, refining 105 a model may involve a variety of steps for personalizing a model which could be used to predict the patient's reaction to a drug used in treatment (e.g., sirolimus). For example, in a case where the disclosed technology was used to implement an application which could be used by a physician to personalize a patient's dose, such an application may determine a particular class of model which may most effectively predict the patient's response. For instance, an application could be configured with a one compartment model, such as described in Mizuno et al., Model-Based Precision dosing of Sirolimus in Pediatric Patients with Vascular Anomalies, EUR. J PHARM. SCI. 2017 Nov. 15; 109S:S124-S131, as well as a two compartment model, such as described in Ferron et al., Population Pharmacokinetics of Sirolimus in Kidney Transplant Patients, CLIN. PHARMACOL. THER. 1997 April; 61 (4): 416-28, and, when refining a patient's dose, may test which of its available model classes more closely matched the collected 103 data for the patient. The model which was found to most closely match the collected 103 data could then be treated as the correct model to use in refining 104 the patient's dose.

Other types of model refinement 105 are also possible. For example, in some cases, equations for predicting the patient's clearance could be updated based on the collected data. For example, in an application implemented based on this disclosure, an existing clearance parameter (which may be an existing clearance parameter for the patient, or, if no such individual clearance parameter is available, may be a population based clearance parameter) may be treated as a prior probability that factors such as food status, fever status, drug-drug interactions, growth and maturation will have an expected effect on clearance, and the application may use Bayesian estimation to obtain an updated parameter which is more closely tailored to the patient's reaction. This type of approach may be used for example, in refining formulas such as equations 1-6, below, which may be used to relate a patient's individual oral clearance of sirolimus (CL/Fᵢ) to the population mean oral clearance for sirolimus (CL/Fₚₒₚ) in light of various factors (e.g., food status, hematocrit, etc.).

$$CL / {F}_{i} = CL / {F}_{pop} \times {( \frac{HCT}{Reference HCT} )}^{{θ}_{HCT}}$$

Equation 1 (in this equation, HCT is hematocrit, and θ_{HCT} represents the effect size of hematocrit on clearance)

$$CL / {F}_{i} = CL / {F}_{pop} \times {θ}_{FOOD}^{FLAG}$$

Equation 2 (in this equation, θ_{FOOD} represents the effect size of the food status, and FLAG is 0 when sirolimus is taken in a faster state or 1 when sirolimus was taken in a fed state)

$$CL / {F}_{i} = CL / {F}_{pop} \times {θ}_{DDI 1}^{FLAG} \times {θ}_{DDI 2}^{FLAG} \times … …$$

Equation 3 (in this equation, FLAG is 0 when the co-medication is not taken, or 1 when the co-medication is taken, and ODDI (n) represents the effect size of co-medication n)

$$CL / {F}_{i} = CL / {F}_{pop} \times {( 1 - θ * {e}^{- K * time} )}^{FLAG}$$

Equation 4 (in this equation, FLAG is 0 when the patient has no fever and 1 when the patient has a fever, time is the time since the onset of fever, θ represents the maximum inverse effect of fever on clearance, and K represents the rate constant to describe the time-dependent recovery of clearance).

$${CL}_{pediatric} = {CL}_{adult} \cdot {( \frac{BW}{70} )}^{Power} \cdot MF$$

Equation 5 (in this equation, CL_{adult} is a clearance in an average adult/older child (70 kg weight).

$$MF = \frac{{PMA}^{Hill}}{{TM}_{50}^{Hill} + {PMA}^{Hill}}$$

Equation 6 (in this equation, MF is a maturation function describing the developmental process, TM₅₀ is the postmenstrual age (PMA) as described in Mizuno et al. Eur J Pharm Sci 2017; 109S:S124-S131 at which 50% of adult clearance is reached, and the Hill coefficient is associated with the slope of the developmental profile).
Of course, it should be understood that the above described approach, in which Bayesian estimation was used to update an existing clearance parameter, is intended to be illustrative, and that other approaches to parameter updating are also possible. For example, in some cases, rather than using Bayesian updating, other types of updates, such as gradient descent updating, could be used instead of the Bayesian updating as described. Accordingly, the above description should not be treated as implying limitations on the scope of protection provided by this document or any related document.

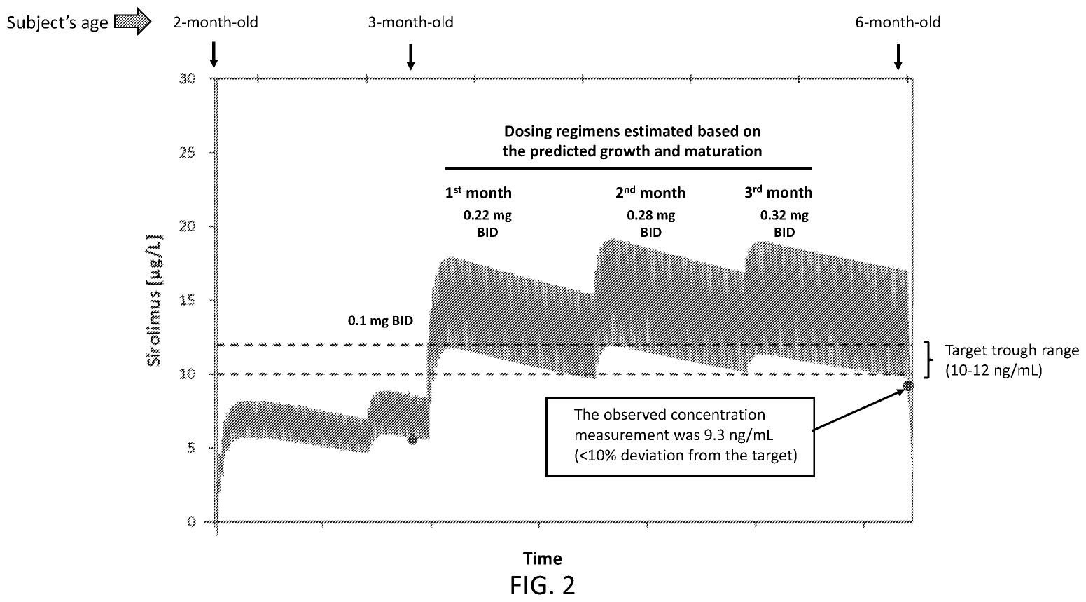

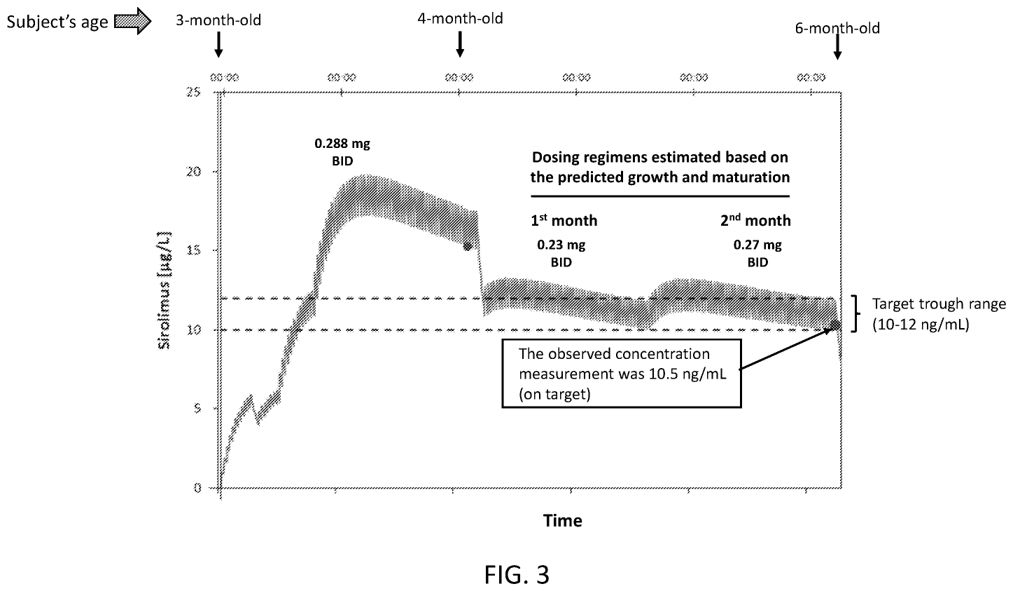

However the model refinement 105 is performed, once a refined model is available, it may be applied 106 to determine a personalized dose for the patient. This may be done, for example, by taking a target trough concentration (e.g., 5-10 ng/ml or 10-15 ng/ml, as shown in table 1), and using the refined model to predict the dose which would be necessary to reach that concentration for the patient. Examples of how an application implemented based on this disclosure may be used to predict, and therefore target, trough concentrations of sirolimus are provided in FIGS. 2 and 3. In those figures, FIG. 2 shows a result of using the disclosed technology to predict sirolimus trough concentrations based on a patient's growth and maturation where the patient is an infant who started sirolimus at 2 months of age. In this example, the sirolimus doses required to maintain the trough concentration in the predefined target range between the ages of 3-6 months were predicted based on the patient's weight, PMA, and a concentration measurement obtained when the patient was three months old. As shown in FIG. 2, the observed concentration at six months of age in this case was 9.3 ng/ml, which was less than a 10% deviation from the target range of 10-12 ng/mL. FIG. 3 provides another example showing how an application implemented based on this disclosure was able to predict the trough concentration between age 4 months to age 6 months base don the concentration measurement at 4 months of age and the patient's weight and PMA. As shown in that figure, the observed concentration at 6 months was 10.5 ng/ml which was within the target trough range of 10-12 ng/mL.

In addition to allowing or precision dosing as described, a refined model may also be used to inform the person who is administering the dose (who may be a caretaker for a pediatric patient, rather than the physician overseeing the treatment) how the dose should be taken. For example, if the refined model was based on data gathered when the patient was in a fed state (see equation 2), then the person administering the dose may not only be informed of an updated amount of the dosed medication, but may also be informed that the medication should be administered when the patient was fed. The personalized dose may then be administered 107 until it is time for additional data to be collected 103 (e.g., after a set period of time, such as one month, has elapsed, or if ongoing monitoring indicates that further refinement in the patient's dose may be necessary). This process may then be repeated, with the model(s) used to determine the patient's dose becoming more and more personalized, until the course of treatment is complete.

To further illustrate how the disclosed technology may be applied in practice, consider a scenario in which the disclosed techniques are used to implement an application which would reside on a server and provide one or more web interfaces to doctors who would use the application to provide personalized sirolimus dosing to pediatric patients. Such an application may, for example, expose a data entry interface. Such an interface may allow a user to provide data regarding the patient, such as an identifier, the patient's date or birth or age, the patient's weight and height, a target exposure and dosing frequency for the patient. This information may then be used by an application to determine an initial dose 102 for the patient, such as by using the entered information, combined with population level statistics, to predict the appropriate sirolimus dose which would result in the target sirolimus concentration as specified using the data entry interface. An application implemented based on this disclosure may also provide a model selection interface, which may be used as part of the model refinement 105 shown in FIG. 1 by allowing a clinician to specify the model that he or she felt was most appropriate for a particular patient. Such an application may also provide a dashboard interface. This type of dashboard interface may provide a recommended refined dose after refining 105 and applying 106 a model used for a patient, as described previously in the context of FIG. 1. Such a dashboard interface may also include data regarding the patient's history with his or her medication, such as a graph showing the concentration of the medication in the patient's blood over the course of treatment as the patient's dose becomes more and more personalized. In this way, an application implemented based on this disclosure may not only assist with personalization of medication but may also be used by a clinician to evaluate the impact that personalization has had on the patient's treatment.

Of course, it should be understood that an application implemented based on this disclosure may include functionality in addition to, or as alternatives to, that described above. For example, in some cases, an application implemented based on this disclosure could generate documentation regarding a patient's treatment, such as by using information provided by a clinician or a laboratory, retrieved from an electronic medical record, or obtained in some other way to complete a consultation form having data such as shown below in table 2.

TABLE 2
Exemplary sirolimus pharmacokinetic consultation form.

PATIENT INFORMATION
Patient Name
Gender
Date of Birth
Gestational age (if start of therapy shortly
after birth)
Weight (most recent) kg
Height (most recent) cm
DOSING DATA
Start of Therapy (date of last dose
adjustment)
Dosing Regimen (mg)
Number of doses per day
Date of Last Rapamune/Sirolimus dose
Time of Last Rapamune/Sirolimus dose
Regular timing of sirolimus doses
In the past 2 days has the dosing varied by
greater than 1 hour from typical timing:
Any missed doses in past 5 days
Any infection in the past few days?
BLOOD LEVEL INFORMATION
Date Sample obtained
Time pre-dose Sample
Time 1 HR-post Sample
Time 3 HR-post Sample
Pre-dose Test Result (ng/ml)
1 HR-post Test Result (ng/ml)
3 HR-post Test Result (ng/ml)
Hematocrit L/L (liter of cells per liter
of blood)
Current Target level (ng/ml)
CONCOMITANT DRUGS
1
2
3
4
5
DOSING RECOMMENDATION PER CLINICIAN
Dose Modification - Dosage (mg)
Dosing Frequency
Target Range
Justification
Comments

Other functionality (e.g., automatically generating patient information sheets, or reminders about side effects) is also possible, and will be immediately apparent to those of skill in the art in light of this disclosure. Similarly, other variations (e.g., using the disclosed technology to predict and generate personalized sirolimus doses for achieving concentrations other than trough concentration). Accordingly, the above description of application functionality should be understood as being illustrative only, and should not be treated as limiting.

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, “based on” should be understood to mean that a thing is determined, at least in part, by that which it is indicated as being “based on.” It should be understood that a statement that something is “based on” something else does not necessarily require one thing to be fully determined by the other. If one thing is required to be fully determined by another, this may be indicated by stating that it is “based EXCLUSIVELY on” that which it is determined by.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

A method of determining and implementing a personalized sirolimus dosing regimen for an individual in need thereof, the method comprising: providing an initial sirolimus dose to the individual; taking a blood sample from the individual; obtaining: a sirolimus concentration for the individual based on the blood sample; and one or more individual-specific clearance parameters for the individual; refining a sirolimus clearance model for the individual based on the sirolimus concentration and at least one of the one or more individual-specific clearance parameters for the individual; using the refined sirolimus clearance model, predicting a personalized sirolimus dose corresponding to achieving a target sirolimus concentration for the individual at a time between three and six months following the refinement of the sirolimus clearance model; and administering the personalized sirolimus dose to the individual.

### Example 2

The method of example 1, wherein refining the sirolimus clearance model for the individual comprises evaluating correspondence between a model selected from: a one compartment model; and a two compartment model; with the individual-specific data.

### Example 3

The method of example 1, wherein the method comprises repeatedly performing the taking, obtaining, refining, using and administering steps through a course of treatment for the individual.

### Example 4

The method of example 1, wherein the sirolimus concentration is a sirolimus trough concentration; and the target sirolimus concentration is a target sirolimus trough concentration.

### Example 5

The method of example 1, wherein the one or more individual specific clearance parameters are selected from a set consisting of: hematocrit; age; co-medications; fever; and maturation function.

### Example 6

The method of example 1, wherein the individual is less than one year old.

### Example 7

The method of example 6, wherein the individual is less than six months old.

### Example 8

A non-transitory computer readable medium having stored thereon instructions programming a computer to perform a set of acts comprising: obtaining: a sirolimus concentration for an individual; and one or more individual-specific clearance parameters for the individual; refining a sirolimus clearance model for the individual based on the sirolimus concentration and at least one of the one or more individual-specific clearance parameters for the individual; using the refined sirolimus clearance model, predicting a personalized sirolimus dose corresponding to achieving a target sirolimus concentration for the individual at a time between three and six months following the refinement of the sirolimus clearance model; and providing an interface comprising the personalized sirolimus dose.

### Example 9

The non-transitory computer readable medium of example 8, wherein refining the sirolimus clearance model for the individual comprises evaluating correspondence between a model selected from: a one compartment model; and a two compartment model; with the individual-specific data.

### Example 10

The non-transitory computer readable medium of example 8, wherein: the sirolimus concentration is a sirolimus trough concentration; and the target sirolimus concentration is a target sirolimus trough concentration.

### Example 11

The non-transitory computer readable medium of example 8, wherein the one or more individual specific clearance parameters are selected from a set consisting of: hematocrit; age; co-medications; fever; and maturation function.

### Example 12

The non-transitory computer readable medium of example 8, wherein the individual is less than one year old.

### Example 13

The non-transitory computer readable medium of example 12, wherein the individual is less than six months old.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of determining and implementing a personalized sirolimus dosing regimen for an individual in need thereof, the method comprising:
a. providing an initial sirolimus dose to the individual;
b. taking a blood sample from the individual;
c. obtaining:
i. a sirolimus concentration for the individual based on the blood sample; and
ii. one or more individual-specific clearance parameters for the individual;
d. refining a sirolimus clearance model for the individual based on the sirolimus concentration and at least one of the one or more individual-specific clearance parameters for the individual;
e. using the refined sirolimus clearance model, predicting a personalized sirolimus dose corresponding to achieving a target sirolimus concentration for the individual at a time between three and six months following the refinement of the sirolimus clearance model; and
f. administering the personalized sirolimus dose to the individual.

2. The method of claim 1, wherein refining the sirolimus clearance model for the individual comprises evaluating correspondence between a model selected from:
a. a one compartment model; and
b. a two compartment model;
with the individual-specific data.

3. The method of claim 1, wherein the method comprises repeatedly performing the taking, obtaining, refining, using and administering steps through a course of treatment for the individual.

4. The method of claim 1, wherein:
a. the sirolimus concentration is a sirolimus trough concentration; and
b. the target sirolimus concentration is a target sirolimus trough concentration.

5. The method of claim 1, wherein the one or more individual specific clearance parameters are selected from a set consisting of:
a. hematocrit;
b. age;
c. co-medications;
d. fever; and
e. maturation function.

6. The method of claim 1, wherein the individual is less than one year old.

7. The method of claim 6, wherein the individual is less than six months old.

8. A non-transitory computer readable medium having stored thereon instructions programming a computer to perform a set of acts comprising:
a. obtaining:
i. a sirolimus concentration for an individual; and
ii. one or more individual-specific clearance parameters for the individual;
b. refining a sirolimus clearance model for the individual based on the sirolimus concentration and at least one of the one or more individual-specific clearance parameters for the individual;
c. using the refined sirolimus clearance model, predicting a personalized sirolimus dose corresponding to achieving a target sirolimus concentration for the individual at a time between three and six months following the refinement of the sirolimus clearance model; and
d. providing an interface comprising the personalized sirolimus dose.

9. The non-transitory computer readable medium of claim 8, wherein refining the sirolimus clearance model for the individual comprises evaluating correspondence between a model selected from:
a. a one compartment model; and
b. a two compartment model;
with the individual-specific data.

10. The non-transitory computer readable medium of claim 8, wherein:
a. the sirolimus concentration is a sirolimus trough concentration; and
b. the target sirolimus concentration is a target sirolimus trough concentration.

11. The non-transitory computer readable medium of claim 8, wherein the one or more individual specific clearance parameters are selected from a set consisting of:
a. hematocrit;
b. age;
c. co-medications;
d. fever; and
e. maturation function.

12. The non-transitory computer readable medium of claim 8, wherein the individual is less than one year old.

13. The non-transitory computer readable medium of claim 12, wherein the individual is less than six months old.
